(19) United States
(12) Patent Application Publication
(10) Pub. No.: US 2002/0120294 A1
(43) Pub. Date: Aug. 29, 2002

(54) Method of implanting a pacemaker

(76) Inventor: Lori C. Kroll, Simi Valley, CA (US)

Correspondence Address: Lori C. Kroll, 493 Sinaloa Road, Simi Valley, CA, 93065, US

(21) Appl. No.: 09/794,882

(22) Filed: Feb. 26, 2001

(51) Int. Cl. A61N 1/00

(52) U.S. Cl. 607/1

(57) ABSTRACT

The invention is the placement of meaningful symbols on the side of a pacemaker housing to give patients greater comfort. The symbols include religious, philosophical, patriotic, or arbitrary themes.

### BACKGROUND OF THE INVENTION

Hundreds of thousands of patients receive a pacemaker every year. While the devices can be lifesaving, the psychological impact of having a foreign object inside the body can be very negative.

In spite of the obvious need for a simple, method for the pacemaker to assist with patient psychological comfort none have been taught. None of the pacemakers manufactured to date have any functionality for serving such a need.

### SUMMARY OF THE INVENTION

The invention is the placement of meaningful symbols on the side of the pacemaker housing to give patients greater comfort. Most people wear at least one piece of jewelry. To have a special symbol directly over the heart and actually connected to it would give great comfort to many patients.

The invention has obvious application to any implantable medical device. This includes but is not limited to implantable cardioverter defibrillators, nerve stimulators, prosthetic hips, etc.

The engraving step could be performed by many equivalent processes such as laser etching, sand blasting, printing, or selectiveanodization. The symbol could also be placed on the insulating header of the pacemaker.

### BRIEF DESCRIPTION OF THE DRAWINGS

FIG. 1: Pacemaker with angel engraving on the side.

FIG. 2: Pacemaker with dove engraving on the side.

FIG. 3: Pacemaker with cross engraving on the side.

FIG. 4: Pacemaker with star of David engraving on the side.

FIG. 5: Pacemaker with fish engraving on the side.

FIG. 6: Method of the invention.

### DETAILED DESCRIPTION OF THE DRAWINGS AND PREFERRED EMBODIMENT

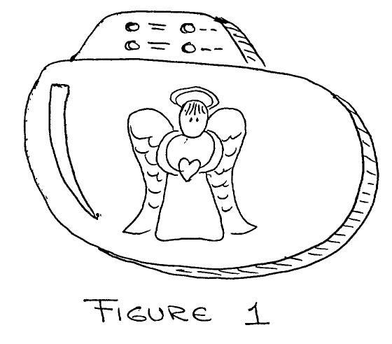

FIG. 1 shows a pacemaker with an angel engraving on the side. This could be comforting to a patient that feels that he or she has a guardian angel.

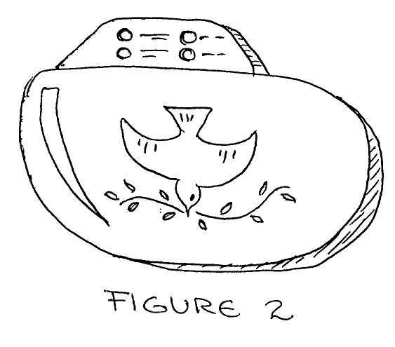

FIG. 2 shows a pacemaker with a dove of peace engraving on the side. This pacemaker could give comfort to many types of patients.

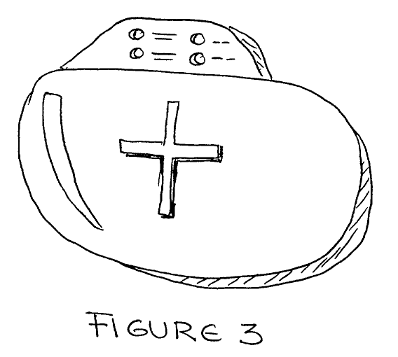

FIG. 3 shows a pacemaker with a cross engraved on the side. This pacemaker would be comforting for patients with a Christian background.

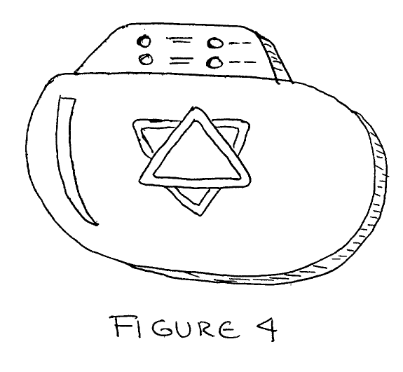

FIG. 4 shows a pacemaker with the star of David on the side. This design would be more popular with patients with a background in Judaism.

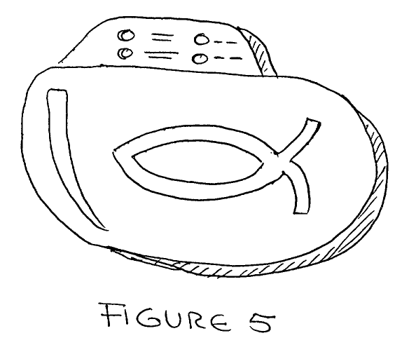

FIG. 5 shows a pacemaker with a fish engraving on the side. This stylized fish is a symbol of Christianity and could be popular for Christians.

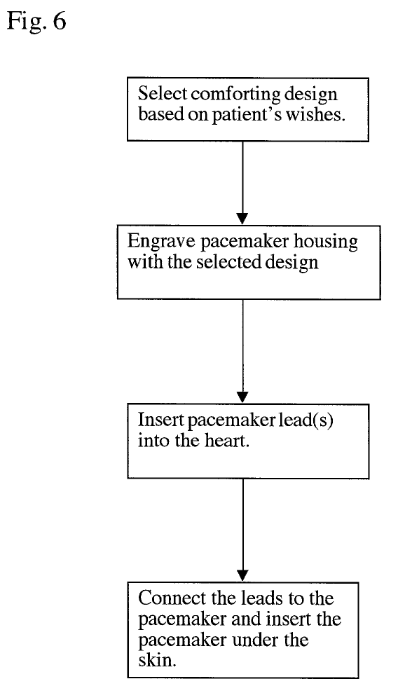

FIG. 6 shows the method of the invention. The first step is, “Select comforting design based on patient's wishes.” The second steep is, “Engrave pacemaker housing with the selected design.” The third step is, “Insert pacemaker lead(s) into the heart.” Finally, the fourth step is, “Connect the leads to the pacemaker and insert the pacemaker under the skin.”

Equivalently, one could keep an inventory of pacemakers on hand with a representative selection of important symbols already engraved on the pacemaker housings.

I claim:

1. A method of implanting a pacemaker comprising the steps of:
selecting a comforting design based on patient's wishes,
adorning the pacemaker housing with the selected design,
inserting at least one pacemaker lead into the heart,
connecting the at least one lead to the pacemaker, and
inserting the pacemaker under the skin.

2. The method of claim 1 in which the pacemaker is oriented so that the symbol will face the heart.

3. The method of claim 1 in which the pacemaker is pre-engraved and the patient selects a symbol from the available inventory.

4. The method of claim 1 in which the pacemaker symbol is a cross.

5. The method of claim 1 in which the pacemaker symbol is an angel.

6. The method of claim 1 in which the pacemaker symbol is a dove of peace.

7. The method of claim 1 in which the pacemaker symbol is a star of David.

8. The method of claim 1 in which the pacemaker symbol is a fish.

9. An implantable pacemaker having a battery, electronic components, and a hermetic housing containing the battery and components with the improvement being the addition of a meaningful symbol to the housing to give psychological comfort to the patient and thus improve the healing process.

10. The pacemaker of claim 9 in which the pacemaker symbol is a cross.

11. The pacemaker of claim 9 in which the pacemaker symbol is an angel.

12. The pacemaker of claim 9 in which the pacemaker symbol is a dove of peace.

13. The pacemaker of claim 9 in which the pacemaker symbol is a star of David.

14. The pacemaker of claim 9 in which the pacemaker symbol is a fish.

15. An implantable medical device having a battery, electronic components, and a hermetic housing containing the battery and components with the improvement being the addition of a meaningful symbol to the housing to give psychological comfort to the patient and thus improve the healing process.

16 The implantable medical device of claim 15 in which the symbol is a cross.

17. The implantable medical device of claim 15 in which the symbol is an angel.

18. The implantable medical device of claim 15 in which the symbol is a dove of peace.

20. The implantable medical device of claim 15 in which the symbol is a star of David.

21. The implantable medical device of claim 15 in which the symbol is a fish.
